(19) United States
(12) Patent Application Publication
(10) Pub. No.: US 2022/0042109 A1
(43) Pub. Date: Feb. 10, 2022

(54) METHODS OF ASSESSING BREAST CANCER USING CIRCULATING HORMONE RECEPTOR TRANSCRIPTS

(71) Applicant: Agendia NV, Amsterdam (NL)

(72) Inventors: Bastiaan Van Der Baan, Irvine, CA (US); Annuska Maria Glas, Irvine, CA (US)

(21) Appl. No.: 17/395,149

(22) Filed: Aug. 5, 2021

Related U.S. Application Data
(60) Provisional application No. 63/062,100, filed on Aug. 6, 2020.

(51) Int. Cl. C12Q 1/6886; G16B 25/10; G16B 40/00

(52) U.S. Cl. CPC C12Q 1/6886; G16B 25/10; C12Q 2600/112; C12Q 2600/158; G16B 40/00

(57) ABSTRACT

The present disclosure provides systems and methods for analyzing circulating hormone receptor transcripts to provide diagnoses, prognoses, and treatment suggestions for patients afflicted with breast cancer. Circulating transcripts can be obtained from patient samples, including blood samples, without the need for invasive tissue biopsies. This may include expression transcripts obtained from extracellular vesicles. Analysis of hormone receptor expression transcripts may include comparison with expression transcripts of patients with known clinical outcomes.

### TECHNICAL FIELD

The present disclosure relates to methods and systems for using machine learning and biomarkers to analyze various conditions, including cancers, such as breast cancer.

### BACKGROUND

Breast cancer is the second most common cancer among women in the United States. Despite advances in screening and treatment, breast cancer remains the second leading cause of cancer death among women. Further, recent studies have shown that there are racial/ethnic variations in breast cancer tumor characteristics, subtypes, relative treatment success rates, and recurrence rates. Moreover, the efficacy of various treatments diverges amongst breast cancer subtypes at various stages of progression. This creates a complex picture for pathologists and oncologists in diagnosing, treating, and predicting recurrence in breast cancer patients. Thus, while the aggregate impact of breast cancer is clear, accurate, patient-specific diagnosis, prognosis, and treatment remains relatively obscured.

Breast cancer is a heterogeneous disease, which was traditionally classed into distinct histological subtypes based on cell morphology. The emergence of various hormone receptors associated with breast cancer led to more granular identification of breast cancer subtypes.

Initially breast cancer was classified according to the status of estrogen receptor (ER). Currently, hormone receptor (HR) status is the gold standard for classifying breast cancer subtypes, which includes the statuses of ER, progesterone receptor (PR), and human epidermal growth factor receptor 2 (HER2 or ERBB2).

HR+/HER2− breast cancers comprise 70% of all cases. HR-negative/HER2− (i.e., triple negative) and HER2+ are less abundant. These clinical subtypes have correlated molecular subtype equivalents. Triple-negative being Basal-type, HER2+ are HER2-type, and HR+ are Luminal-type. Luminal-type breast cancer is further subdivided into A and B subtypes, which differ based upon expression of the protein Ki67.

These subtypes require different treatments and result in vastly different clinical outcomes. HER2-type are more aggressive and result in generally worse prognoses compared to Luminal-type. Basal-type generally provide the most dire prognoses.

Unfortunately, breast cancer cannot always simply be defined according to HR± status. For instance, breast cancer can also present as ER±/PR±. Patients with single-HR+ breast cancers generally have an intermediate prognoses between Luminal-type and Basal-type.

A further complicating factor is that other hormone receptors have been linked to breast cancer. For example, high androgen receptor (AR) expression in some forms of breast cancer indicates a positive prognosis, while in other types has been shown to be a marker for breast cancer proliferation. Expression levels of epidermal growth factor receptor (EGFR), G-protein-coupled estrogen receptor 1 (GPER1), fibroblast growth factor receptors (FGFR), and many other receptors have been linked to breast cancer outcomes.

Accordingly, there is a need to better measure the expression levels of hormone receptors to better characterize breast cancers in order to provide targeted treatments and improve clinical outcomes.

### SUMMARY

The invention relates to methods and systems for analyzing breast cancer by analyzing the expression levels of one or more hormone receptors based on circulating transcripts (e.g., RNA) from patient samples. The systems and methods of the disclosure allow for breast cancer to be more accurately subtyped and diagnosed in particular patients and patient groups. This can inform treatment decisions and better assess clinical outcomes, such as the risk of recurrence or metastasis.

Unlike previous methods reliant on obtaining tissue samples from tumors, the present disclosure provides systems and methods that do not require tissue biopsies from breast cancer tumors to obtain hormone receptor status. Rather, fluid samples, such as a simple blood draw, can provide the necessary circulating transcripts for analysis.

This shift in sample type confers several advantages to the systems and methods of the present disclosure. By forgoing tissue biopsies, patients experience less discomfort when providing samples for testing. Further, because the systems and methods of the invention are not reliant on tissue samples from tumors they can be used in the context of routine screening. This allows potential diagnoses even the in the absence of evident tumor formation. It also broadens the scope of patients who can be tested.

For example, many women only receive breast cancer testing after a potential malignancy is detected from self-examinations or mammograms. Clearly, self-examination is inconsistent and mammograms are often performed only on women over the age of 40. As a result, many breast cancers are not detected until they are already at an advanced stage. By using the methods and systems of the disclosure, minimally-invasive, routine breast cancer screenings can be performed in a wide variety of settings. This can allow for early detection of breast cancer, especially in younger patients, and dramatically improve clinical outcomes for this time-sensitive disease.

Additionally, since tumor tissue is not required for testing, patients can be evaluated before and after a tumor excision, allowing more accurate prognosis and diagnosis of recurrence. This allows physicians to more properly assess whether a particular patient will benefit from aggressive treatment after tumor removal. Similarly, the methods and systems of the disclosure may assess breast cancer in a patient undergoing treatment and determine whether the patient is responding to a particular treatment and whether additional or more aggressive treatments, such as chemotherapy, would prove beneficial.

Further, breast cancer is often diagnosed using tissue sample for histopathology assays. However, histopathology relies upon subjective analysis, visual perception, and the judgment of individual pathologists in order to form a diagnosis. Thus, even when viewing the same tissue sample, different pathologists can form different diagnoses. By using circulating transcripts, consistent and objective diagnoses can be obtained without reliance on the “human factor” prevalent in histopathology. The ability of the methods and systems of the disclosure to accurately ascertain the subtype and status of a patient's breast cancer ensure that the most targeted treatments are provided.

The methods of the disclosure include methods for analyzing breast cancer that include determining an expression level for one or more hormone receptors or hormone receptor subtypes from transcripts in circulation in a sample obtained from a patient. This may include preparing a sample to specifically isolate and enrich circulating hormone receptor expression transcripts. Then, using said expression level to diagnose and/or stage breast cancer when said expression level is above a predetermined threshold. Predetermined thresholds may be formed based on data from prior patients whose transcript expression levels have been correlated to particular diagnoses or prognoses.

The methods and systems of the disclosure can also be used to create or analyze one or more hormone receptor expression signatures indicative of breast cancer subtype, stage, and/or clinical outcome. The expression signatures may be correlated with expression signatures of known patient outcomes. In turn, the expression signatures can be used to provide tailored treatments for individual patients or patient groups. For example, methods of the invention may be used to identify a patient who may safely avoid the toxicities of chemotherapy and/or may be used to guide a course of treatment by identifying a drug that will be effective for treating breast cancer associated with a particular expression signature.

The methods and systems of the disclosure may comprise determining quantitative amounts circulating of expression transcript RNA a sample and determining a ratio of different species of the transcripts RNA. Such ratios can themselves be used as thresholds in the methods and systems of the disclosure. Ratios may also form parts of expression signatures.

The methods and systems of the disclosure may obtain circulating expression transcripts from extracellular vesicles obtained from a patient. RNA expression transcripts in extracellular vesicles, whether from cancer or non-cancer cells, can be more stable and less biased than those obtained and isolated from cells, including tumor cells. This means that expression transcripts derived from extracellular vesicles can provide a more faithful representation of expression in cancer cells. This is especially beneficial in the methods and systems of the disclosure in which a patient's expression transcripts are monitored over time. This allows, for example, physicians to ascertain the efficacy of treatments, recurrence, and remission. Further, as breast cancer is heterogeneous in nature, using the systems and methods of the invention, a patient can be monitored over time to determine whether expression transcripts are indicative that the breast cancer has morphed into a more or less severe type. Thus, the method may further comprise isolating an extracellular vesicle from the sample and determining the expression level using the contents of the vesicle.

Further, the systems and methods of the disclosure may be employed at least several months after diagnosis of breast cancer to detect or predict a risk of recurrence of the breast cancer. Such an analysis may be based upon expression signatures of patients who have known outcomes.

The methods and systems of the disclosure may also comprise measuring or detecting the levels of one or more circulating hormones in conjunction with circulating transcript expression levels. Advantageously, both levels may be measured from a single sample. Analysis of hormones and hormone receptor expression can lead to more accurate diagnoses and prognoses. Analysis of both hormones and hormone receptors can be used in a confirmatory manner, or as part of a signature.

The methods and systems of the disclosure may also include analyzing circulating expression transcripts in a sample for hormone receptors and for one or more additional marker genes. Analyzing the circulating transcript levels of hormone receptors and marker genes, the systems and methods of the invention can provide accurate prognoses and diagnoses. Although hormone receptors are known to play a critical role in breast cancer, other marker genes have been correlated with types and prognoses of various breast cancers.

Such marker genes may include, for example: AA555029_RC; ALDH4A1; AP2B1; AYTL2; BBC3; C16orf61; C20orf46; C9orf30; CCNE2; CDCl42BPA; CDCA7; CENPA; COL4A2; DCK; DIAPH3; DTL; EBF4; ECT2; EGLN1; ESM1; EXT1; FGF18; FLT1; GMPS; GNAZ; GPR126; GPR180; GSTM3; HRASLS; IGFBP5; JHDM1D; KNTC2; LGP2; LIN9; LOC100131053; LOC100288906; LOC730018; MCM6; MELK; MMP9; MS4 A7; MTDH; NMU; NUSAP1; ORC6L; OXCT1; PALM2; PECI; PITRM1; PRC1; QSCN6L1; RAB6B; RASSF7; RECQL5; RFC4; RTN4RL1; RUNDC1; SCUBE2; SERF1A; SLC2A3; STK32B; TGFB3; TSPYL5; UCHL5; WISP1; and ZNF533. In some methods and systems of the disclosure, substantially all of these marker genes are analyzed in conjunction with circulating hormone receptor expression.

Marker genes may be measured with probes and/or panels. Thus, the determining step of methods and systems of the disclosure may also comprise interrogating a sample with probes for substantially all of a panel, and measuring the expression levels for positive probe responses.

The methods and systems of the disclosure may also include analyzing an image of tissue from the patient to support or confirm the diagnosis. The image may comprise a digital scan of a stained, FFPE slide from a tumor from the patient.

The methods and systems of the disclosure may employ machine learning to analyze circulating transcript expression of hormone receptors. Machine learning may be deployed at many points during the methods of the invention. For example, machine learning may be used in measuring the transcript expression levels and/or when analyzing the transcript levels. By using machine learning, the methods and systems of the invention can find new correlations, for example, between hormone receptor expression levels and particular diagnoses, prognoses, and treatment efficacies. Using the power of machine learning, the methods and systems of the invention can leverage vast amounts of old and/or new data to provide more accurate and patient-specific diagnoses, prognoses, and treatment suggestions.

Thus, the methods and systems of the disclosure may include providing hormone receptor transcript expression levels as inputs to an analysis system trained on training data comprising one or more sets of training expression level measurements associated with known outcomes. Further, other data, such as hormone levels, marker gene expression, image data, etc. from the subject may be provided as part of the inputs to the analysis system. The methods and systems of the disclosure can analyze this disparate data, such as receptor transcript levels and image data, in combination to provide correlative diagnoses, prognoses, and treatment suggestions. The methods and systems of the disclosure may include a computer system hosting a trained machine learning algorithm. Image data provided as an input may be an image of a stained, FFPE slide from a tumor from the patient or an image of a microarray.

### BRIEF DESCRIPTION OF DRAWINGS

FIG. 1 shows a workflow according to the disclosure.

FIG. 2 a body fluid sample according to the disclosure.

FIG. 3 sample preparation according to the disclosure.

FIG. 4 shows a machine learning workflow according to the disclosure.

FIG. 5 shows a platform of the disclosure.

FIG. 6 shows a computer system.

### DETAILED DESCRIPTION

The present disclosure relates to methods and systems for analyzing breast cancer using expression levels of hormone receptors. Expression levels are obtained using samples derived from circulating transcripts, e.g., RNA transcripts from circulating tumor cells (ctRNA), cell-free RNA transcripts (cfRNA), and RNA transcripts from extracellular vesicles, such as exosomes.

Advantageously, because the disclosure uses circulating transcripts, it is not always necessary to perform an invasive biopsy to obtain a tissue sample from a tumor. Preferably, the circulating transcripts can be obtained from bodily fluids such as blood. Not only can this reduce patient discomfort, but can increase access to testing. This can allow for more universal long-term monitoring and facilitate longitudinal study. As a result, the systems and methods of the disclosure can provide more accurate diagnoses and prognoses. Similarly, the relatively simple procedures for obtaining and analyzing samples allows for more data to be derived, which over time can make the disclosed methods and systems more accurate and individualized.

Further, using bodily fluids as a sample source rather than tumor tissue, patients can be evaluated before and after a breast cancer tumor is removed. This allows, for example, a physician to determine whether the tumor is likely to recur and/or metastasize. Further, such testing can be leveraged for use as routine screening to diagnose early-stage breast cancer and prior to detecting breast cancer tumors.

Such insights can guide treatment options, e.g., whether a patient will benefit from one or more rounds of chemotherapy. In other instances, the methods and systems of the disclosure may assess breast cancer in a patient undergoing chemotherapy and determine whether the patient is responding to a particular treatment and whether additional or more aggressive treatments, such as chemotherapy, would prove beneficial. Further, the ability of the methods and systems of the disclosure to accurately ascertain the subtype and status of a patient's breast cancer ensure that the most targeted treatments are provided.

The disclosure includes not only methods using measured expression levels of hormones or hormone receptors, but additional data, such as image data, expression levels of one or more marker genes, and hormone measurements. The systems and methods of the disclosure allow for breast cancer to be more accurately subtyped and diagnosed in particular patients and patient groups. This can inform treatment decisions and better assess clinical outcomes, such as the risk of recurrence or metastasis.

The methods and systems of the disclosure can be used to create one or more hormone or hormone receptor expression signatures indicative of breast cancer subtype, stage, and/or clinical outcome. The expression signatures may be correlated with expression signatures of known patient outcomes. In turn, the expression signatures can be used to provide tailored treatments for individual patients or patient groups.

For example, methods of the invention may be used to identify a patient who may safely avoid the toxicities of chemotherapy and/or may be used to guide a course of treatment by identifying a drug that will be effective for treating breast cancer associated with a particular expression signature.

The systems and methods of the disclosure can predict how well a given patient will respond to certain treatments. As the systems and methods of the invention are useful for predicting how well a patient will respond to certain treatments, an effective treatment may be recommended to the patient, and clinicians can avoid spending the time and money on treatment protocols that will not help the patient. Further, because the systems and methods of the disclosure can use samples obtained from bodily fluids, testing and analysis is far more rapid than existing tests. Consequently, physicians can quickly administer an appropriate and effective treatment. This helps improve the prognoses of patients with early-stage breast cancer.

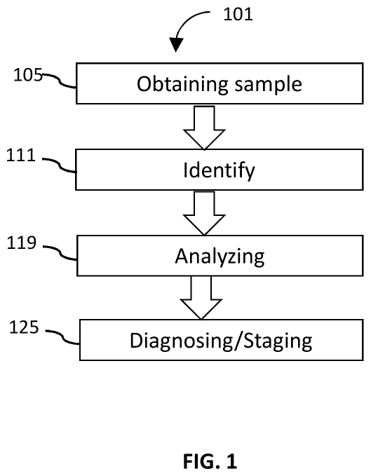

FIG. 1 diagrams a general workflow 101 employed by the methods and systems of the disclosure. The workflow requires obtaining 105 a sample from a patient comprising circulating transcripts. The sample is assayed to identify expression levels 111 of hormone receptors and/or receptor subtypes. Then, the expression levels of relevant hormone receptors or hormone receptor subtypes are analyzed 119 to determine, for example, whether they are above or below predetermined threshold levels. Then, a patient's breast cancer is diagnosed and/or staged 125, for example, based upon whether the expression levels for relevant hormone receptors or receptor subtypes fall above or below the predetermined threshold levels.

When more than one receptor or receptor subtype expression levels are determined, the expression levels can form an expression signature. In such instances, analyzing 119 can comprise a comparison with predetermined expression signatures, that each comprise predetermined expression levels for the hormone receptors and/or receptor subtypes. The predetermined expression signatures can correspond to a particular breast cancer diagnosis, stage, subtype, prognosis, etc., and used to diagnose/stage the patient's breast cancer.

Analyzing 119 may also include determining a quantitative amount of each relevant transcript assayed. Further, the quantitative amounts of each transcript assayed can be used to form ratios (e.g., the quantitative expression of hormone receptor 1 to the quantitative expression of hormone receptor 2). Such ratios may be compared to predetermined threshold ratios, used as an expression signature, and/or used as part of a larger expression signature.

The methods and systems of the disclosure may identify 111 circulating expression transcripts of any hormone receptor thought implicated with breast cancer. These can include, by way for example, estrogen receptors (ER), progesterone receptors (PR), human epidermal growth factor receptor 2 (HER2 or ERBB2), androgen receptor follicle stimulating hormone (FSH) receptors, epidermal growth factor receptors (EGFR), G-protein-coupled estrogen receptor 1 (GPER1), and fibroblast growth factor receptors (FGFR). Circulating expression transcripts for hormone receptor subtypes can also by analyzed by the methods and systems of the disclosure. For example, in ER, subtypes can include ERα and ERβ. The methods and systems of the disclosure can also analyze expression transcripts of hormone receptor isoforms. For example, in ER, isoforms can include ERN, ERN-short form, ERβ2, ERβ2/cx, ERβ3, ERβ4, ERβ5, and the like.

Accordingly, the methods and systems of the disclosure can identify 111 circulating expression levels of hormone receptors, quantify the expression levels, and determine ratios between the expression levels of hormone receptors, receptor subtypes, and/or receptor isoforms. These ratios can be compared with predetermined thresholds, including as part of an expression signature.

Circulating expression transcripts identified 111 by the methods and systems of the disclosure may include cell-free RNA (cfRNA), which may include, for example, messenger RNA (mRNA), microRNA (miRNA), long non-coding RNA (lncRNA), and circular RNA (circRNA). Preferably, the cell-free RNA is obtained 105 from extracellular vesicles, such as exosomes. Both healthy cells and breast cancer cells may transcribe, process, and package expressed RNA in extracellular vesicles. These vesicles have been implicated as part of cellular cross-talk. Importantly, extracellular vesicles may provide a more stable environment for transcripts. Not only does this provide more complete transcripts for analysis, but it presents a more accurate representation of cellular activity than lysing cells to release transcripts. The systems and methods of the disclosure leverage this feature of extracellular vesicles to provide a more meaningful analysis of a patient's current breast cancer status.

Circulating transcripts can be obtained 105 from body fluid samples. Body fluid samples can comprise one of blood, saliva, sputum, urine, semen, transvaginal fluid, cerebrospinal fluid, sweat, stool, a cell or a tissue. Preferably, the sample comprises blood or serum, as it is an insight of the invention that circulating transcripts are surprisingly stable in blood when encapsulated inside extracellular vesicles where they are protected from degradation.

Accordingly, the method 101 may further involve segregating extracellular vesicles from a patient's blood sample and subsequently isolating circulating transcripts from the segregated vesicles prior to the identifying 111 step. The body fluid sample may be obtained 105 from a patient suspected of having breast cancer or determined to be at risk for breast cancer. However, due to the relatively non-invasive nature of the disclosed methods and systems, the body fluid sample may be obtained from a patient undergoing routine screening for breast cancer. Alternatively, the patient may be suspected of having breast cancer due to the presentation of symptoms associated with breast cancer, e.g., the detection of a lump or mass. More preferably, the cancer is early stage breast cancer, i.e., cancer that is contained entirely within the breast. However, the methods and systems of the disclosure can be used during and after a patient's course of treatment for breast cancer. This allows treatment methodologies to be assessed and changed based on the response of the breast cancer to treatment, as indicated by the systems and methods of the disclosures.

The hormone receptor transcripts may be identified 111, e.g., detected and/or quantified, by any of a wide variety of methods known in the art. For example, sequencing (e.g., RNA-seq), hybridization analysis (e.g., microarray), amplification e.g., via the polymerase chain reaction, for example, by reverse transcription polymerase chain reaction (RT-PCR/RT-qPCR).

Preferably, identifying 111 involves targeted enrichment and creation of cDNA libraries from RNA transcripts, and using next-generation sequencing technologies to sequence and quantitate the transcripts. For example, identifying 111 can involve isolating cell free nucleic circulating transcripts from a patient, such as transcripts comprising cfRNA. The cfRNA is converted into complementary DNA (cDNA). Specific cDNA molecules associated with hormone receptors of interest are probed for using biotinylated capture RNA baits. The captured cDNA molecules are then identified 111 by sequencing to produce a plurality of sequence reads which are compared to one or more reference genomes to identify the origin of the cDNA molecule. The cDNA can include barcodes. Barcodes can be used to quantify the expression levels of each hormone receptor transcript.

In the methods and systems of the disclosure, expression levels of hormone transcripts may be used to provide predictive values. Predictive values may indicate the probability, based on expression levels, that a patient has breast cancer or a particular subtype of breast cancer and/or the clinical risk associated with a patient's breast cancer. For example, a predictive value may indicate the probability that a patient's breast cancer has a high risk of recurrence or a distant metastatic event. The predictive value may include a component based on time. For example, the risk of recurrence or metastasis within a defined time period, e.g., within five or ten years. Predictive values can also be associated with disease severity and staging.

Predictive values may be based on correlating certain circulating hormone receptor transcript levels, ratios, and/or signatures with those of patients with known outcomes. Thus, analyzing 119 may include comparing a patient's transcript levels, ratios, and/or signatures, with those of patients with known outcomes. This may include weighting certain hormone receptor transcript levels, with a bias towards those hormone receptors shown to have stronger correlations with particular diagnoses, prognoses, clinical outcomes, and the like.

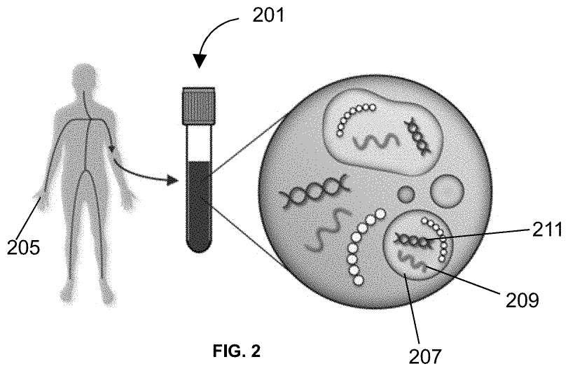

FIG. 2 shows a body fluid sample 201. The body fluid sample 201 comprises blood 203 and preferably taken from a patient 205 by blood draw. The blood 203 contains extracellular vesicles 207, which are small plasma membrane-encapsulated particles released from all cells that can enter into the bloodstream. Extracellular vesicles 207 comprise exosomes and micro-vesicles. Exosomes are small extracellular vesicles (50-100 nanometers) of endocytic origin while micro-vesicles are larger particles (100-1,000 nanometers) that are shed via direct cell membrane budding.

Extracellular vesicles 207 contain proteins (tumor antigens, immunosuppressive, and/or angiogenic molecules) and cell free nucleic acids, including cell-free RNA 209 and cell-free DNA 211 specific to cancer cells. Thus, their cargo may be analyzed to determine their cell of origin by, for example, by segregating the extracellular vesicles 207 and sequencing the nucleic acids contained therein or performing an immunochemistry staining for cell-type specific proteins. In some cases, the extracellular vesicles 207 may be segregated by immunostaining the extracellular vesicles 207 for a protein that is over or under expressed in cancer, and subsequently sorting the stained extracellular vesicles 207 by FACS. Accordingly, methods of the invention may include the step of determining an extracellular vesicle's origin (e.g., determining that the vesicle was released from a tumor cell) based on the content of the extracellular vesicle before identifying the circulating hormone receptor transcripts contained therein.

By determining the extracellular vesicle's origin prior to identifying the cell free nucleic acids, a research or clinician, may focus their analyses specifically on nucleic acids associated with breast cancer, such as hormone receptor transcripts. Extracellular vesicles 207 are ubiquitous in body fluids including plasma, cerebral spinal fluid, aqueous humor, amniotic fluid, saliva, synovial fluid, adipose tissue, and urine. Both plasma and cerebral spinal fluid extracellular vesicles including exosomes are a useful source of cell free nucleic acids for assessing disease. Accordingly, methods of the invention allow for the analysis of extracellular vesicle cargo, to track and predict tumor growth and allow early treatment for patients. Alternatively, patients with treatment-related pseudo-progression may be spared unnecessary and potentially ineffective changes in treatment strategy.

The body fluid sample may be collected by blood draw or by fine needle aspiration and the hormone receptor transcripts extracted from extracellular vesicles, such as exosomes, present in the blood sample. Isolating the extracellular vesicles from the body fluid sample may be required. To isolate extracellular vesicles from the body fluid sample a method of differential ultracentrifugation (low-speed centrifugation to remove cells and debris, high-speed ultracentrifugation to pellet exosomes) may be performed. For example, to isolate extracellular vesicles from a blood the sample, the sample may be centrifuged at low speeds allowing for the removal of cells and debris by, for example, pipetting or dumping out supernatant. The sample may then be centrifuged at high speeds, for example, at 100,000×g for 70 min, to pellet the extracellular vesicles allowing the extracellular vesicles to be separated from remaining material. Easy-to-use precipitation solutions, such as the precipitation solution sold under the trade name ExoQuick by System Biosciences, may be used to precipitate the vesicles in liquid. Once the vesicles are isolated, the vesicles may be lysed in lysis buffer to release the cell free nucleic acids. For example, as described Garcia, 2019, Isolation and Analysis of Plasma-Derived Exosomes in Patients With Glioma, Front Oncol, 9: 651, incorporated by reference.

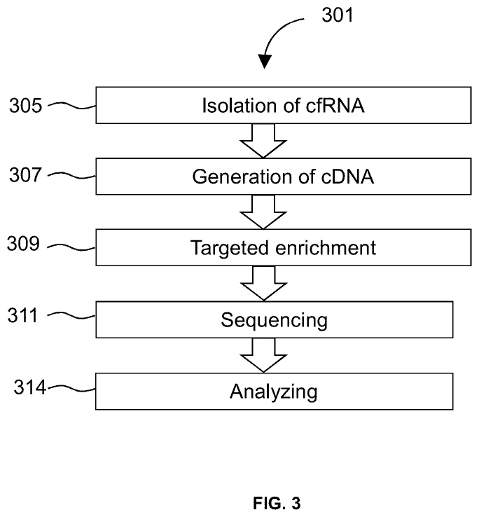

FIG. 3 diagrams a method 301 of sample preparation according to certain methods and systems of the disclosure. The method 301 includes isolating 305 cfRNA. The cfRNA is preferably isolated from extracellular vesicles collected in a blood sample. In some embodiments, RNA isolation 305 is performed with an RNA isolation kit sold, such as the RNA isolation kit sold under the trade name RNeasy by Qiagen (Valencia, Calif.), and in accordance with the manufacturer's instructions. Isolated cfRNA preferably has a 260/280 and 260/230 absorbance ratio values close to 2.0. To determine the quality of the RNA, a nucleic acid analysis system, such as the Agilent 2100 Bioanalyzer instrument, may be used. The cfRNA may be chemically fragmented. Preferably, the fragments comprise 200 base pairs.

Following isolation 305, the cfRNA is converted to cDNA. The generation of cDNA 307 can be done by a variety of methods, but, preferably, the cDNA is generated using reverse transcriptase, which has the ability to use the information in a molecule of RNA to generate a molecule of cDNA. Reverse transcriptase is a RNA-dependent DNA polymerase. Like all DNA polymerases it cannot initiate synthesis de novo but depends on the presence of a primer. Since many RNAs have a poly-A tail at the 3′ end, oligo-dT is frequently used to prime DNA synthesis. It is also possible, and frequently essential, to generate cDNAs by using either random primers or primers designed to amplify a specific RNA. Once a first strand of cDNA has been created, it is generally necessary to produce a second strand of DNA. A person of skill in the art will recognize that there are many methods for producing the second strand, but a convenient mechanism involves exposure of the DNA/RNA hybrid to a combination of RNAase-H and DNA polymerase. RNAase-H has the ability to cause single-stranded nicks in the RNA, and DNA polymerase can then use these single-stranded nicks to initiate “second strand” DNA synthesis. This two-step procedure has been optimized to maximize fidelity and length of cDNAs.

Adapters may be ligated onto the ends of the cDNA. The cDNA may be adenylated at the 3′ end prior to adapter ligation. Preferably, the adapters comprise sequencing platform specific primers, such as the Illumina P5/P7 (flow cell binding primers). The adapters may also comprise PCR primer biding sites for amplifying the cDNA library. In some embodiments, the adapters may further include barcode sequences. The barcode sequences may be used to give each molecule of cDNA a unique tag, e.g., a unique molecular identifier. Unique molecular identifiers or molecular barcodes are short DNA molecules which may be ligated onto DNA fragments, e.g., cDNA fragments. The random sequence composition of the unique molecular identifiers assures that every fragment-unique molecular identifier's combination is unique in the library. Thus, after PCR amplification, it is possible to distinguish multiple copies of a fragment caused by PCR clones versus real biological duplications. By using unique molecular identifiers, PCR clones can be found by searching for non-unique fragment-UMI combinations, which can only be explained by PCR clones. Following adapter ligation, the cDNA may be amplified by PCR.

Biotinylated capture baits or probes are can be used for the targeted enrichment 309 of specific cDNA molecules of interest. The biotinylated capture probes may comprise RNA, DNA, or a hybrid of RNA and DNA nucleotides. Preferably, the capture probes comprise biotinylated RNAs, which provide better signal to noise ratios. The biotinylated RNA capture probes may be added to the cDNA library and incubated for a period of time and at a temperature sufficient for the biotinylated RNA capture probes to hybridize to their target molecules of cDNA based on Watson-Crick base pairing. For example, the mixture containing cDNA and probes may be incubated at 65 degrees Celsius for 24 hours. After hybridization, the biotinylated RNA capture probes that are hybridized with the target cDNA molecules may be captured and segregated using streptavidin or an antibody. The target cDNA molecules can then be amplified by PCR.

The library may then be sequenced 311. An example of a sequencing technology that can be used is Illumina sequencing. Illumina sequencing is based on the amplification of DNA on a solid surface using fold-back PCR and anchored primers. Genomic DNA is fragmented and attached to the surface of flow cell channels. Four fluorophore-labeled, reversibly terminating nucleotides are used to perform sequential sequencing. After nucleotide incorporation, a laser is used to excite the fluorophores, and an image is captured and the identity of the first base is recorded. Sequencing according to this technology is described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, each incorporated by reference. In preferred embodiments, an Illumina Mi-Seq sequencer is used. The Ilumina Mi-Seq sequencer is used to generate a plurality of sequence reads that may be uploaded to a web portal for analysis by, for example, the Agendia Data Analaysis Pipeline Tool (ADAPT).

Analyzing 314 the sequence reads may be performed using known software and following a multistep procedure known in the art. For example, first, the quality of each sequence read, i.e., FASTQ sequence, may be assessed using the software FASTQC. Next, the reads may be trimmed by, for example, Trimmomatic software. The trimmed sequence reads may then be mapped to a human genome using the HISAT2 software. HISAT2 output files in a SAM (sequence alignment/map format), which may be compressed to binary sequence alignment/map files using SAMtools version prior sequence read quantification. Afterward, mapped reads may be counted using the feature Counts software.

The methods and systems of the disclosure may analyze levels of circulating hormones in conjunction with analyzing circulating hormone receptor expression. As with receptor expression, the levels of circulating hormones may be quantified. The levels of various hormones may be used to provide a hormone signature. Hormone signatures and hormone receptor expression signatures may be combined to form a hormonal milieu signature. Hormonal levels may be compared to predetermined thresholds and/or signatures.

Hormones can be analyzed from the same sample used to derive a hormone receptor expression. This may include obtaining hormone levels and hormone expression levels from extracellular vesicles in a sample.

Hormone levels can be determined by any methods known in the art, including but not restricted to, immunoabsorbant assays, enzyme-linked immunoabsorbant assays, chromatography, gas chromatography, mass spectrometry, and/or liquid chromatography-electrospray ionization tandem mass spectrometry.

Any relevant circulating hormones may be analyzed in accordance with the methods and systems of the disclosure. Additionally, circulating hormone metabolites and hormone precursors can be analyzed in by the disclosed methods and systems. Especially important are circulating sex hormones/steroids, including androgens, estrogens, progestogens, testosterones, and the like. Important hormones/metabolites/precursors/steroids implicated in breast cancer, and analyzed by the systems and methods of the invention can include progesterone, estradiol (hormone estrogen), testosterone, prolactin, oxysterols, pregnenolone, insulin-like growth factor 1 (IGF1), IGF-binding protein 3, sex hormone-binding blobuline (SHBG), 5α-dihydroprogesterone (5αP), 3α-dihydroprogesterone (3αP), 17-hydroxypregnenolone, 17-hydroxyprogesterone, 20α-dihydroprogesterone, allopregnanedione, allopregnanolone, dehydroepiandrosterone, androstenedione, androstenedione, androsterone, androstenediol, dihydotestosterone, andostanediol, 2-hydroxyestrone, estrone, 16α-hydroxyestrone, 2-hydroxyestradiol, estradiol, estriol, estetrol, 27-hydroxycholesterol, anti-Müllerian hormone, luteinizing hormone.

A further key biomarker feature used by the systems and method of the disclosure is imaging data, such as histopathology data, e.g., whole-slide imaging (WSI). WSI has long been used to diagnose breast cancer, including subtypes, stage, and prognoses. By combining image data with hormone receptor transcript expression levels, a more accurate and complete picture of a patient's breast cancer can be produced.

This finds particular use in longitudinal monitoring of patients/tumors. For example, a tissue sample may be extracted from a patient's tumor, and the extraction imaged (e.g., using WSI). Then, the tissue sample is assayed for circulating hormone receptor transcript expression data. This process can be iterated over time and/or over different areas of the tumor, to determine, for example, the tumor's response to treatment, to assess the heterogeneous nature of the tumor, to find one or more subtypes of cancer associated with the tumor, and to find the major biologic driver/drivers of the tumor.

Image data can be obtained from tissue samples. Tissue samples may comprise tissue slices harvested from a patient. The tissue slices may contain information regarding the pathological status of the tissue. Alternatively, the image data may comprise images of cells collected by, for example a biopsy, and deposited onto a slide. The cells may include any human cell type, such as, for example, lymphocytes, erythrocytes, macrophages, T-cells, skin cells, fibroblasts, epithelial cells, blood cells, etc.

In the methods and systems of the disclosure WSI, several features may be assessed, for example, the spatial arrangements and architecture of different types of tissue elements. This can include, by way of example, global features of the epithelial and stromal regions, diversity of nuclear shape, orientation, texture, and architecture, glandular architecture, tumor infiltrating lymphocytes, lymphocyte proximity to cancer cells, the ratio of intratumoural lymphocytes to cancer cells, the tumor stroma, etc.

In addition to analyzing the levels of circulating hormone receptor expression transcripts, RNA expression of other genes can be analyzed in accordance with the methods and systems of the invention. Such transcripts are known to be an important biomarker feature analyzed to diagnose and predict clinical outcomes of diseases and conditions, including breast cancer. RNA expression levels of several genes have been shown to correlate specific disease types and probable clinical outcomes.

For example, the BluePrint test (Agendia®) is an 80-gene signature assay that measures the combined RNA expression of 80 genes. This test has consistently been able to classify the majority of tested breast cancer patients into definitive breast cancer clinical subtypes, i.e., Luminal-type, Basal-type, and HER2-type. (Mittempergher et al., Translational Oncology, 13 (2020) 100756). For each clinical subtype, a signature RNA expression was determined. A patient's RNA profile is compared to these signature RNA expression levels to determine the patient's clinical subtype. Id. The MammaPrint test (Agendia®) is a 70-gene signature assay that measures the combined RNA expression of 70-genes to assign breast cancer tumors as being of a high or low risk for metastasis. Id. These tests guide a physician's treatment decisions, including whether to pursue early chemotherapy, and avoiding aggressive treatments when they would provide no benefit.

The systems and methods of the disclosure can use machine learning (ML) in conjunction with levels of circulating hormone expression transcripts to analyze breast cancer. This includes, not only providing a diagnosis or prognosis based on known expression transcript signatures, but also creating novel correlations between expression transcripts and other data.

Machine learning is branch of computer science in which machine-based approaches are used to make predictions. (Bera et al., Nat Rev Clin Oncol., 16(11):703-715 (2019)). ML-based approaches involve a system learning from data fed into it, and use this data to make and/or refine predictions. Id. Machine learning is distinct from traditional, rule-based or statistics-based program models. (Rajkomar et al., N Engl J Med, 380:1347-58 (2019)). Rule-based program models require software engineers to code explicit rules, relationships, and correlations. Id. For example, in the medical context, a physician may input a patient's symptoms and current medications into a rule-based program. In response, the program will provide a suggested treatment based upon preconfigured rules.

In contrast, and as a generalization, in ML a model learns from examples fed into it. Id. Over time, the ML model learns from these examples and creates new models and routines based on acquired information. Id. As a result, an ML model may create new correlations, relationships, routines or processes never contemplated by a human. A subset of ML is deep learning (DL). (Bera et al. (2019)). DL uses artificial neural networks. A DL network generally comprises layers of artificial neural networks. Id. These layers may include an input layer, an output layer, and multiple hidden layers. Id. DL has been shown to learn and form relationships that exceed the capabilities of humans. (Rajkomar et al. (2019)).

By combining the ability of ML, including DL, to develop novel routines, correlations, relationships and processes amongst vast data sets of disease biomarker features and patients' clinical data features, the methods and systems of the disclosure can provide accurate diagnoses, prognoses, and treatment suggestions tailored to specific patients and patient groups afflicted with diseases, including breast cancer.

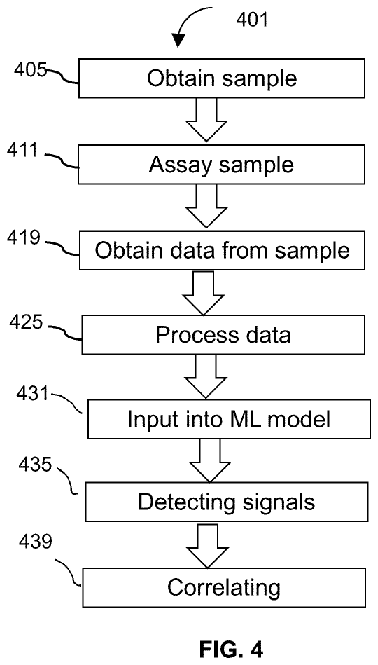

FIG. 4 diagrams a general workflow used in the methods and systems of the present disclosure. The workflow 401 includes obtaining sample 405 from a patient relevant to a particular disease, such as breast cancer. For example, a sample may include a tissue biopsy, a blood draw, and the like. This may include obtaining more than one type of sample from a patient, e.g., a blood draw for a circulating hormone receptor transcript expression analysis and a tissue biopsy for a histopathology analysis. The sample is assayed 411. For example, tissue biopsies may be prepared and stained for a whole-slide image (WSI) and blood draws may be used to isolate and sequence nucleic acids to determine RNA expression levels. Then, relevant data is obtained 419 for any assay completed.

Once data is obtained 419, the data is processed 425. Processing the data 425 transforms the data into signals that can be analyzed by an ML model. For example, if a whole-slide image was obtained, the image is transformed into pixels that can be analyzed by an ML model. Processing the data 425 may also include normalizing or tuning the data. For example, with a WSI, the color and saturation of the image can be adjusted to account for differences arising between imaging instruments or staining procedures. Processing the data 425 may further include annotation. Annotation may, for example, comprise indicating or identifying certain features or areas of interest on a WSI. Annotation may also include clinical feature data relevant to a particular patient, such as age, sex, gender, ethnicity and the like. These annotations may be used by the ML model.

Processing the data 425 may be performed by one or more relevant algorithms and/or by human interaction. Processing 425 may be iterative, such that the data undergoes multiple rounds or methods of processing to fine-tune the data.

After processing the data 425 it is in a format that can be used by an ML model, and includes signals of clinical features that the model analyzes. The processed data is then input into the ML model 431. The ML model analyzes the data to detect relevant signals 435. Detecting signals may include, for example, identifying certain biomarker features, such as hormone receptor expression levels and the spatial distribution of immune cells on a WSI. The ML model correlates these signals 439 to provide a predictive output. The predictive output may be a predictive diagnosis, prognosis, assignment to a particular risk category, a treatment suggestion, and the like. Based on this predictive output a clinician may undertake an appropriate action, such as a particular course of treatment or non-treatment, monitoring, or subsequent testing.

Predictive outputs may include a metric for each type of biomarker feature analyzed. These metrics may be combined to form a larger prediction. These metrics may be weighted. Predictive outputs may include signature biomarker features for certain types of conditions, for example, a hormone receptor transcript expression signature for a subtype of breast cancer. Predictive outputs may be used to assess disease severity, such as staging breast cancer or predicting the risk of metastasis, recurrence, or residual risk. Predictive outputs may be longitudinal. Longitudinal outputs may be outputs for the same patient or patient population over time, and updated based upon additional biomarker feature data or clinical feature data. Predictive outputs may be based upon threshold values for one or more biomarker features or clinical features. Threshold values may be created by ML models or by humans. ML models may be used to provide predictive outputs for various treatment options for particular patients or patient populations. A single blood draw or tissue sample may be used to provide biomarker feature data to provide predictive outputs for a patient's risk (e.g., likely risk of metastasis for a tumor), relative treatment efficacies, and benefit of further monitoring (e.g., how often the patient should have a tumor analyzed). A patient's breast cancer may be monitored at several time points, by obtaining samples over time to be analyzed by an ML model to provide continual predictive outputs, including a risk score and treatment score.

Using machine learning, the systems and methods of the disclosure can expand and improve diagnostic accuracy and prognoses based upon circulating hormone receptor expression levels.

For example, patients' circulating hormone receptor transcript expression profiles and clinical feature data are input into an ML model. Clinical feature data includes, for example, patients' age, sex, ethnicity, comorbidities, treatments, changes in expression over time and in response to treatment, and clinical outcomes, including recurrence. The ML model learns from these data sets and creates novel correlations amongst biomarker feature data and clinical feature data. For example, a correlation between a particular expression profile and the presence/likelihood of a particular form of breast cancer. These novel correlations are used to create predictive outcomes, which may be more accurate for specific patients and patient subgroups. This improves the diagnostic and prognostic value of existing circulating hormone receptor transcript expression tests.

Advantageously, the more patients' that have their hormone receptor expression levels analyzed, the more data is available for ML learning inputs. Larger data sets can naturally include, for example, expression profiles taken during different stages of disease progression and profiles taken after patients have undergone varying treatment regimes. Leveraging the correlative power of ML and its ability to digest and learn from vast data sets, diagnostic and prognostic predications become more patient-specific, and thus accurate for individuals.

Additionally, when using established tests, data sets with consistent formats can be created and shared amongst various physicians and researchers. For example, data sets may be provided as electronic case record forms (eCRF). Individual eCRF files may have their information extracted and placed into a Trial Master File (TMF). These data sets may be tailored in such a way to provide ML input data relevant to investigative and clinical trials.

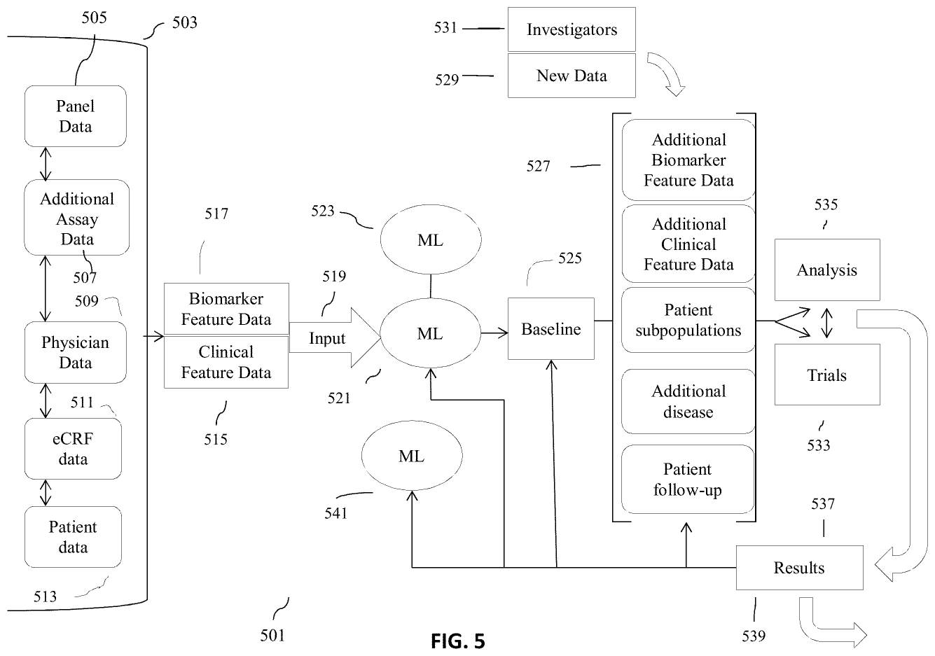

FIG. 5 provides a general overview of a platform of the disclosure through which data, for example, from an established panel to detect circulating hormone receptor transcript expression, can be leveraged using ML to improve and/or expand the established panel, or be used to create new tests and discoveries.

The platform 501 receives data 503 from several sources, which can include biomarker feature data 517 and clinical feature data 517 from patients. Biomarker feature data from patients may come from an established panel 505 or one or more additional assays 507. This data may be received directly into the platform from, for example, an instrument that processed an established panel. The data may likewise come from physicians 509, such as in the form of an electronic medical record. The data may also come from studies/trials, investigators, researchers and the like, such as in the form of eCRFs 511. Patients may also provide data 513.

Clinical feature data 515, such as patients' age, sex, ethnicity, comorbidities, clinical outcomes, medical treatments and history, patients' familial histories, etc., may be provided by, for example, physicians 509, eCRFs 511, and patients 513, such as through the use of surveys.

Clinical feature data 515 and biomarker feature data 517 are prepared as inputs 519 for an ML model 521. Preparing as inputs 519 may include, for example, processing, normalizing, and annotating the data. This data may be used as data sets to train the ML model 521, or be analyzed by the ML model to provide predictive results. The ML model may train or analyze the data using one or more additional ML models 523. The ML model 521 creates a baseline 525 for providing predictive results. From this baseline 525, patient subsets 527 may emerge or be derived. The patient subsets 527 may include emerge or be derived based upon, for example, relevant new data 529 or actions by one or more investigators 531.

Patient subsets 527 may include, for example, subsets according to additional biomarker feature data (e.g., new hormone receptor expression data), additional clinical feature data (e.g., groups of patients undergoing a specific treatment modality), patient subpopulations (e.g., groups of patients having similar ages, ethnicities, comorbidities, etc.), additional diseases/conditions (e.g., patient groups investigated for the purpose of investigating a disease/condition or who develop a particular disease condition), and patient follow-up (e.g., survey questions, longitudinal monitoring, clinical outcomes, biomarker feature data/clinical feature data over time, etc.). The investigation or emergence of the patient subsets 527 may lead to new trials/studies 533 or analysis 535. Trials/studies 533 and/or analysis 535 may lead to further trials/studies 533 and/or analysis 535. From trials/studies and/or analysis, results 537 can emerge. Results 537 may leave the platform 539, for example, as published studies. Results 537, may also be used to determine further patient subsets 527, update the baseline 525, used as an input to the ML model 521 for analysis and/or training, or put into a different ML model 541 as an input for analysis and/or training.

Further, as circulating hormone receptor transcript expression profiling becomes faster and more ubiquitous, ML can be leveraged to expand existing panels or create new panels. For example, using clinical data inputs and expanded expression profiles, ML can create novel correlations between newly significant genes and clinical data. Similarly, expression profiles from single cells, cellular components, and extracellular components, such as exosomes, can provide more patient-specific predictive correlations. For example, extracellular vesicles have more stable RNA expression profiles relative cells themselves. This is especially important in the heterogeneous and ever-changing environment of a tumor.

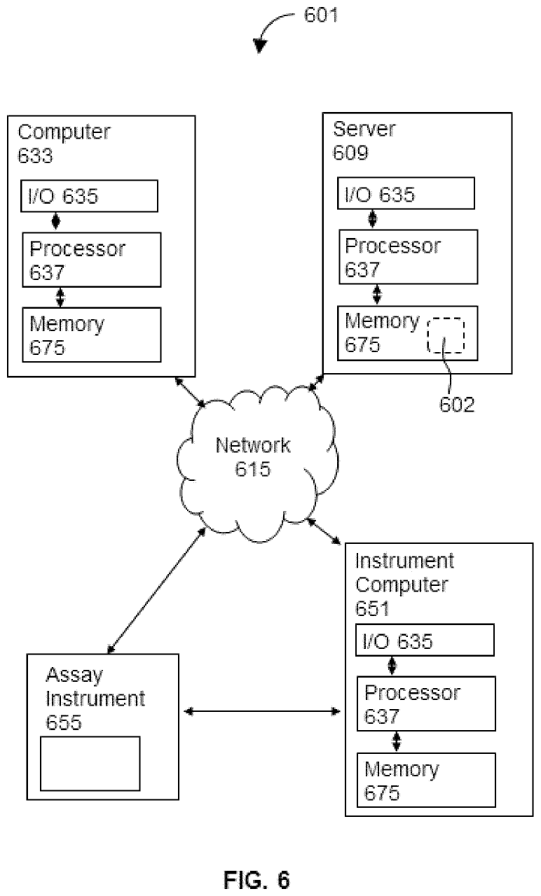

FIG. 6 shows a computer system 601 that may include a machine learning subsystem 602 that has been trained on training data sets. In preferred embodiments, the machine learning subsystem performs the detecting 435. The system 601 includes at least one processor 637 coupled to a memory subsystem 675 including instructions executable by the processor 637 to cause the system 601 to detect 435 relevant signals; and to determine 439 a correlation to provide a predictive output.

The system 601 includes at least one computer 633. Optionally, the system 601 may further include one or more of a server computer 609 one or more assay instruments 655 (e.g., a microarray, nucleotide sequencer, an imager, etc.), which may be coupled to one or more instrument computers 651. Each computer in the system 601 includes a processor 637 coupled to a tangible, non-transitory memory 675 device and at least one input/output device 635. Thus, the system 601 includes at least one processor 637 coupled to a memory subsystem 675. The components (e.g., computer, server, instrument computers, and assay instruments) may be in communication over a network 615 that may be wired or wireless and wherein the components may be remotely located or located in close proximity to each other. Using those mechanical components, the system 201 is operable to receive or obtain training data such (e.g., images and molecular assay data) and outcome data as well as test sample data generated by one or more assay instruments or otherwise obtained. The system may use the memory to store the received data as well as the machine learning system data which may be trained and otherwise operated by the processor.

Processor refers to any device or system of devices that performs processing operations. A processor will generally include a chip, such as a single core or multi-core chip (e.g., 12 cores), to provide a central processing unit (CPU). In certain embodiments, a processor may be a graphics processing unit (GPU) such as a NVidia Tesla K80 graphics card from NVIDIA Corporation (Santa Clara, Calif.). A processor may be provided by a chip from Intel or AMD. A processor may be any suitable processor such as the microprocessor sold under the trademark XEON E5-2620 v3 by Intel (Santa Clara, Calif.) or the microprocessor sold under the trademark OPTERON 6200 by AMD (Sunnyvale, Calif.). Computer systems of the invention may include multiple processors including CPUs and or GPUs that may perform different steps of methods of the invention.

The memory subsystem 675 may contain one or any combination of memory devices. A memory device is a mechanical device that stores data or instructions in a machine-readable format. Memory may include one or more sets of instructions (e.g., software) which, when executed by one or more of the processors of the disclosed computers can accomplish some or all of the methods or functions described herein. Preferably, each computer includes a non-transitory memory device such as a solid state drive, flash drive, disk drive, hard drive, subscriber identity module (SIM) card, secure digital card (SD card), micro SD card, or solid-state drive (SSD), optical and magnetic media, others, or a combination thereof.

Using the described components, the system 601 is operable to produce a report and provide the report to a user via an input/output device. An input/output device is a mechanism or system for transferring data into or out of a computer. Exemplary input/output devices include a video display unit (e.g., a liquid crystal display (LCD) or a cathode ray tube (CRT)), a printer, an alphanumeric input device (e.g., a keyboard), a cursor control device (e.g., a mouse), a disk drive unit, a speaker, a touchscreen, an accelerometer, a microphone, a cellular radio frequency antenna, and a network interface device, which can be, for example, a network interface card (NIC), Wi-Fi card, or cellular modem. The machine learning subsystem 602 has preferably trained on training data that includes training images and known marker quantities.

Machine learning systems of the invention may be configured to assay data, and known outcomes, to identify features within assay data in an unsupervised manner and to create a map of outcome probabilities over features in the assay data. The machine learning system can further receive assay data from a test subject, identify within the assay data predictive features learned from the training steps and locate the predictive features on the map of outcome probabilities to provide a prognosis or diagnosis.

Any of several suitable types of machine learning may be used for one or more steps of the disclosed methods. Suitable machine learning types may include neural networks, decision tree learning such as random forests, support vector machines (SVMs), association rule learning, inductive logic programming, regression analysis, clustering, Bayesian networks, reinforcement learning, metric learning, and genetic algorithms. One or more of the machine learning approaches (aka type or model) may be used to complete any or all of the method steps described herein.

For example, one model, such as a neural network, may be used to complete the training steps of autonomously identifying features and associating those features with certain outcomes. Once those features are learned, they may be applied to test samples by the same or different models or classifiers (e.g., a random forest, SVM, regression) for the correlating steps. In certain embodiments, features may be identified and associated with outcomes using one or more machine learning systems and the associations may then be refined using a different machine learning system. Accordingly some of the training steps may be unsupervised using unlabeled data while subsequent training steps (e.g., association refinement) may use supervised training techniques such as regression analysis using the features autonomously identified by the first machine learning system.

In decision tree learning, a model is built that predicts that value of a target variable based on several input variables. Decision trees can generally be divided into two types. In classification trees, target variables take a finite set of values, or classes, whereas in regression trees, the target variable can take continuous values, such as real numbers. Examples of decision tree learning include classification trees, regression trees, boosted trees, bootstrap aggregated trees, random forests, and rotation forests. In decision trees, decisions are made sequentially at a series of nodes, which correspond to input variables. Random forests include multiple decision trees to improve the accuracy of predictions. See Breiman, 2001, Random Forests, Machine Learning 45:5-32, incorporated herein by reference. In random forests, bootstrap aggregating or bagging is used to average predictions by multiple trees that are given different sets of training data. In addition, a random subset of features is selected at each split in the learning process, which reduces spurious correlations that can results from the presence of individual features that are strong predictors for the response variable. Random forests can also be used to determine dissimilarity measurements between unlabeled data by constructing a random forest predictor that distinguishes the observed data from synthetic data. Id.; Shi, T., Horvath, S. (2006), Unsupervised Learning with Random Forest Predictors, Journal of Computational and Graphical Statistics, 15(1):118-138, incorporated herein by reference. Random forests can accordingly by used for unsupervised machine learning methods of the invention.

SVMs are useful for both classification and regression. When used for classification of new data into one of two categories, such as having a disease or not having the disease, a SVM creates a hyperplane in multidimensional space that separates data points into one category or the other. Although the original problem may be expressed in terms that require only finite dimensional space, linear separation of data between categories may not be possible in finite dimensional space. Consequently, multidimensional space is selected to allow construction of hyperplanes that afford clean separation of data points. See Press, W. H. et al., Section 16.5. Support Vector Machines. Numerical Recipes: The Art of Scientific Computing (3rd ed.). New York: Cambridge University (2007), incorporated herein by reference. SVMs can also be used in support vector clustering to perform unsupervised machine learning suitable for some of the methods discussed herein. See Ben-Hur, A., et al., (2001), Support Vector Clustering, Journal of Machine Learning Research, 2:125-137.

Regression analysis is a statistical process for estimating the relationships among variables such as features and outcomes. It includes techniques for modeling and analyzing relationships between multiple variables. Specifically, regression analysis focuses on changes in a dependent variable in response to changes in single independent variables. Regression analysis can be used to estimate the conditional expectation of the dependent variable given the independent variables. The variation of the dependent variable may be characterized around a regression function and described by a probability distribution. Parameters of the regression model may be estimated using, for example, least squares methods, Bayesian methods, percentage regression, least absolute deviations, nonparametric regression, or distance metric learning.

Association rule learning is a method for discovering interesting relations between variables in large databases. See Agrawal, 1993, Mining association rules between sets of items in large databases, Proc 1993 ACM SIGMOD Int Conf Man Data p. 207, incorporated by reference. Algorithms for performing association rule learning include Apriori, Eclat, FP-growth, and AprioriDP. FIN, PrePost, and PPV, which are described in detail in Agrawal, 1994, Fast algorithms for mining association rules in large databases, in Bocca et al., Eds., Proceedings of the 20th International Conference on Very Large Data Bases (VLDB), Santiago, Chile, September 1994, pages 487-499; Zaki, 2000, Scalable algorithms for association mining, IEEE Trans Knowl Data Eng 12(3):372-390; Han, 2000, Mining Frequent Patterns Without Candidate Generation, Proc 2000 ACM SIGMOD Int Conf Management of Data; Bhalodiya, 2013, An Efficient way to find frequent pattern with dynamic programming approach, NIRMA Univ Intl Conf Eng, 28-30 Nov. 2013; Deng, 2014, Fast mining frequent itemsets using Nodesets, Exp Sys Appl 41(10):4505-4512; Deng, 2012, A New Algorithm for Fast Mining Frequent Itemsets Using N-Lists, Science China Inf Sci 55(9): 2008-2030; and Deng, 2010, A New Fast Vertical Method for Mining Frequent Patterns, Int J Comp Intel Sys 3(6):333-344, the contents of each of which are incorporated by reference. Inductive logic programming relies on logic programming to develop a hypothesis based on positive examples, negative examples, and background knowledge. See Luc De Raedt. A Perspective on Inductive Logic Programming. The Workshop on Current and Future Trends in Logic Programming, Shakertown, to appear in Springer LNCS, 1999; Muggleton, 1993, Inductive logic programming: theory and methods, J Logic Prog 19-20:629-679, incorporated herein by reference.

Bayesian networks are probabilistic graphical models that represent a set of random variables and their conditional dependencies via directed acyclic graphs (DAGs). The DAGs have nodes that represent random variables that may be observable quantities, latent variables, unknown parameters or hypotheses. Edges represent conditional dependencies; nodes that are not connected represent variables that are conditionally independent of each other. Each node is associated with a probability function that takes, as input, a particular set of values for the node's parent variables, and gives (as output) the probability (or probability distribution, if applicable) of the variable represented by the node. See Charniak, 1991, Bayesian Networks without Tears, AI Magazine, p. 50, incorporated by reference.

In preferred embodiments, the machine learning subsystem 602 uses a neural network. Preferably, the machine learning subsystem 602 includes a deep-learning neural network that includes an input layer, an output layer, and a plurality of hidden layers.

A neural network, which is modeled on the human brain, allows for processing of information and machine learning. The neural network includes nodes that mimic the function of individual neurons, and the nodes are organized into layers. The neural network includes an input layer, an output layer, and one or more hidden layers that define connections from the input layer to the output layer. The neural network may, for example, have multiple nodes in the output layer and may have any number of hidden layers. The total number of layers in a neural network depends on the number of hidden layers. For example, the neural network may include at least 5 layers, at least 10 layers, at least 15 layers, at least 20 layers, at least 25 layers, at least 30 layers, at least 40 layers, at least 50 layers, or at least 100 layers. The nodes of the neural network serve as points of connectivity between adjacent layers. Nodes in adjacent layers form connections with each other, but nodes within the same layer do not form connections with each other. The neural network has an input layer, n hidden layers, and an output layer. Each layer may comprise a number of nodes.

The system may include any neural network that facilitates machine learning. The system may include a known neural network architecture, such as GoogLeNet (Szegedy, et al. Going deeper with convolutions, in CVPR 2015, 2015); AlexNet (Krizhevsky, et al. Imagenet classification with deep convolutional neural networks, in Pereira, et al. Eds., Advances in Neural Information Processing Systems 25, pages 1097-3105, Curran Associates, Inc., 2012); VGG16 (Simonyan & Zisserman, Very deep convolutional networks for large-scale image recognition, CoRR, abs/3409.1556, 2014); or FaceNet (Wang et al., Face Search at Scale: 90 Million Gallery, 2015), each of the aforementioned references are incorporated by reference.

Training data includes data relevant to the assay data which the neural network will analyze, which may be annotated with known outcomes. Nodes in the input layer receive assay data from one or more individuals. For example, the nodes may receive circulating hormone receptor expression data. The known outcomes associated with the training images are provided to the neural network.

Deep learning (also known as deep structured learning, hierarchical learning or deep machine learning) is a class of machine learning operations that use a cascade of many layers of nonlinear processing units for feature extraction and transformation. Each successive layer uses the output from the previous layer as input. The algorithms may be supervised or unsupervised and applications include pattern analysis (unsupervised) and classification (supervised). Certain embodiments are based on unsupervised learning of multiple levels of features or representations of the data. Higher level features are derived from lower level features to form a hierarchical representation. Those features can be represented within nodes as feature vectors.

Deep learning by the neural network includes learning multiple levels of representations that correspond to different levels of abstraction; the levels form a hierarchy of concepts. In most preferred embodiments, the neural network includes at least 5 and preferably more than 10 hidden layers. The many layers between the input and the output allow the system to operate via multiple processing layers.

Deep learning is part of a broader family of machine learning methods based on learning representations of data. An observation (e.g., an image) can be represented in many ways such as a vector of intensity values per pixel, or in a more abstract way as a set of edges, regions of particular shape, etc. Those features are represented at nodes in the network. Preferably, each feature is structured as a feature vector, a multi-dimensional vector of numerical features that represent some object. The feature provides a numerical representation of objects, since such representations facilitate processing and statistical analysis. Feature vectors are similar to the vectors of explanatory variables used in statistical procedures such as linear regression. Feature vectors are often combined with weights using a dot product in order to construct a linear predictor function that is used to determine a score for making a prediction.

The vector space associated with those vectors may be referred to as the feature space. In order to reduce the dimensionality of the feature space, dimensionality reduction may be employed. Higher-level features can be obtained from already available features and added to the feature vector, in a process referred to as feature construction. Feature construction is the application of a set of constructive operators to a set of existing features resulting in construction of new features.

Within the network, nodes are connected in layers, and signals travel from the input layer to the output layer. The nodes of the hidden layer may be calculated as a function of a bias term and a weighted sum of the nodes of the input layer, where a respective weight is assigned to each connection between a node of the input layer and a node in the hidden layer. The bias term and the weights between the input layer and the hidden layer are learned autonomously in the training of the neural network. The network may include thousands or millions of nodes 3 and connections. Typically, the signals and state of artificial neurons are real numbers, typically between 0 and 1. Optionally, there may be a threshold function or limiting function on each connection and on the unit itself, such that the signal must surpass the limit before propagating. Back propagation is the use of forward stimulation to modify connection weights, and is sometimes done to train the network using known correct outputs.

The systems and methods of the disclosure may use convolutional neural networks (CNN). A CNN is a feedforward network comprising multiple layers to infer an output from an input. CNNs are used to aggregate local information to provide a global predication. CNNs use multiple convolutional sheets from which the network learns and extracts feature maps using filters between the input and output layers. The layers in a CNN connect at only specific locations with a previous layer. Not all neurons in a CNN connect. CNNs may comprise pooling layers that scale down or reduce the dimensionality of features. CNNs hierarcially deconstruct data into general, low-level cues, which are aggregated to form higher-order relationships to identify features of interest. CNNs predictive utility is in learning repetitive features that occur throughout a data set.

The systems and methods of the disclosure may use fully convolutional networks (FCN). In contrast to CNNs, FCNs can learn representations locally within a data set, and therefore, can detect features that may occur sparsely within a data set.

The systems and methods of the disclosure may use recurrent neural networks (RNN). RNNs have an advantage over CNNs and FCNs in that they can store and learn from inputs over multiple time periods and process the inputs sequentially.

The systems and methods of the disclosure may use generative adversarial networks (GAN), which find particular application in training neural networks. One network is fed training exemplars from which it produces synthetic data. The second network evaluates the agreement between the synthetic data and the original data. This allows GANs to improve the prediction model of the second network.

The outcome data may include information related to a disease or condition. For example, and without limitation, the outcome data may include information on one or more of breast cancer tumor metastasis, tumor growth, or patient survival related to breast cancer. The outcome data is from one or more individuals from whom other data, e.g., circulating hormone receptor expression data have been or will be entered into the machine learning system. In various embodiments the training sets may include data from patients that are cancer free and the machine learning system may identify features that differentiate between cancer positive and cancer free tissues.

The features detected by the machine learning system may be any quantity, structure, pattern, or other element that can be measured from the training data. Features may be unrecognizable to the human eye. Features may be created autonomously by the machine learning system. Alternatively, features may be created with user input.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of diagnosing breast cancer, the method comprising the steps of
determining an expression level for one or more hormone receptors or hormone receptor subtypes from transcripts in circulation in a sample obtained from a patient; and
utilizing said expression level to diagnose and/or stage breast cancer when said expression level is above a predetermined threshold.

2. The method of claim 1, further comprising measuring or detecting one or more hormones in the sample and using the expression level and hormone information in the diagnosis.

3. The method of claim 1, wherein said utilizing step comprises determining a quantitative amount of said transcripts and determining a ratio of different species of said RNA.

4. The method of claim 1, wherein the transcripts comprise cell-free RNA in the sample.

5. The method of claim 1, further comprising isolating an extracellular vesicle from the sample and determining the expression level for contents of the vesicle.

6. The method of claim 1, wherein the determining step comprises measuring the transcripts in the sample for the hormone receptors and for one or more additional marker genes.

7. The method of claim 6, wherein the marker genes are selected from a panel comprising: AA555029_RC; ALDH4A1; AP2B1; AYTL2; BBC3; C16orf61; C20orf46; C9orf30; CCNE2; CDCl42BPA; CDCA7; CENPA; COL4A2; DCK; DIAPH3; DTL; EBF4; ECT2; EGLN1; ESM1; EXT1; FGF18; FLT1; GMPS; GNAZ; GPR126; GPR180; GSTM3; HRASLS; IGFBP5; JHDM1D; KNTC2; LGP2; LIN9; LOC100131053; LOC100288906; LOC730018; MCM6; MELK; MMP9; MS4 A7; MTDH; NMU; NUSAP1; ORC6L; OXCT1; PALM2; PECI; PITRM1; PRC1; QSCN6L1; RAB6B; RASSF7; RECQL5; RFC4; RTN4RL1; RUNDC1; SCUBE2; SERF1A; SLC2A3; STK32B; TGFB3; TSPYL5; UCHL5; WISP1; and ZNF533.

8. The method of claim 7, wherein the determining step comprises interrogating the sample with probes for substantially all of the panel, and measuring the expression levels for positive probe responses.

9. The method of claim 7, further comprising performing the recited steps at least several months after diagnosis of breast cancer to detect or predict a risk of recurrence of the breast cancer.

10. The method of claim 1, further comprising analyzing an image of tissue from the patient to support or confirm the diagnosis.

11. The method of claim 10, wherein the image comprises a digital scan of a stained, FFPE slide from a tumor from the patient.

12. The method of claim 1, wherein the utilizing step comprises providing the determined expression levels as inputs to an analysis system trained on training data comprising one or more sets of training expression level measurements associated with known outcomes.

13. The method of claim 12, wherein the analysis system comprises a computer system hosting a trained machine learning algorithm.

14. The method of claim 12, further comprising providing image data from the subject as part of the inputs to the analysis system, wherein the analysis system performs an analysis on a combination of the image data and the expression levels.

15. The method of claim 14, wherein the image data comprises an image of a stained, FFPE slide from a tumor from the patient or an image of a microarray.

16. The method of claim 1, wherein the sample is a blood sample.
